(19) United States
(12) Patent Application Publication
(10) Pub. No.: US 2010/0119655 A1
(43) Pub. Date: May 13, 2010

(54) MUSCULAR PROTEIN DENATURATION INHIBITOR, ADDITIVE FOR A FISH MEAT-ORIGIN GROUND MEAT, FISH MEAT-ORIGIN GROUND MEAT CONTAINING THE SAME AND METHOD OF PRODUCING THE SAME

(75) Inventors: Koichi Kuwahara, Nagasaki (JP); Kunihiko Konno, Hokkaido (JP)

Correspondence Address: QUINN EMANUEL;KODA & ANDROLIA, 865 S. FIGUEROA STREET, 10TH FLOOR, LOS ANGELES, CA, 90017, US

(73) Assignee: Nagasaki Prefectural Government

(21) Appl. No.: 12/452,782

(22) PCT Filed: Jul. 24, 2008
(86) PCT No.: PCT/JP2008/063279

§ 371 Date: Jan. 22, 2010

(30) Foreign Application Priority Data
Jul. 25, 2007 (JP) 2007-193221

(51) Int. Cl. A23B 4/12; C07C 55/22; A23B 4/07

(52) U.S. Cl. 426/63; 562/590

(57) ABSTRACT

To provide a muscular protein denaturation inhibitor which is effective only in a small addition amount because of exerting an excellent effect of inhibiting heat denaturation and freeze denaturation of fish meat-origin muscular proteins, shows almost no taste as a solution, has been approved as a food additive, also has been employed as a component in supplements due to its health-promoting effects, and is safe to the human body. A muscular protein denaturation inhibitor characterized by containing a citric acid salt as the active ingredient and being capable of inhibiting heat denaturation and/or freeze denaturation of fish meat-origin muscular proteins.

### TECHNICAL FIELD

The present invention relates to a muscular protein denaturation inhibitor having an effect of significantly inhibiting heat denaturation and freeze denaturation of fish meat-origin muscular proteins, an additive for a fish meat-origin ground meat, a fish meat-origin ground meat containing the same and a method of producing the same.

### BACKGROUND ART

Generally, fish meat-origin ground products are thermally-solidified gelled food by forming a fish paste obtained by adding salt to a fish meat and further adding seasoning, etc. thereto to grind the fish meat and dissolve proteins therein. In principle, fish meat-origin ground products are manufactured by uniformly dissolving muscular proteins in the fish meat, such as myosin, with salt to convert them into a paste and heating the paste to cause heat denaturation and form gel networks therein.

Currently, production of fish meat-origin ground products particularly employs a frozen ground meat. The frozen ground meat, which is advantageous in its treatment in large volumes and stable fish prices, is produced in many parts of the world, and commonly known as “SURIMI.”

Gel strength is one of the most important factors for the quality of ground product, and mechanical weakness of a gel is attributed to non-uniform networks. More specifically, even a single deficiency from such non-uniform networks is prone to stress concentration therein, resulting in gel destruction. In order to produce high-quality ground meat products (both frozen and unfrozen), it is significantly important to completely and uniformly dissolve muscular proteins in a fish meat without heat denaturation therein.

However, a conventional method of producing a ground meat poses a major problem of frictional heat in the processes of collecting a fish meat, removing contaminants therefrom and grinding the fish meat, resulting in heat denaturation of muscular proteins therein. Unfortunately, this heat denaturation fails to completely and uniformly dissolve muscular proteins in the fish meat and produce elastic heat-induced gels, having a poorer quality of ground products. Likewise, since a conventional method of producing a frozen ground meat subjects a ground meat to a freezing treatment, fish meat-origin muscular proteins are prone to denaturation, resulting in a poorer quality of ground products as well.

To solve this problem, sugars are conventionally used as an inhibitor against heat denaturation and freeze denaturation of proteins, particularly sorbitol (sugar alcohol) as an inhibitor against denaturation of fish meat-origin muscular proteins. For example, 4 to 8 weight % of sorbitol is added to frozen ground meat mainly from Theragra chalcogramma to provide 1-year frozen storage period.

Meanwhile, production of fish meat-origin ground products employs salt to completely dissolve fish meat-origin muscular proteins. Salt addition can completely dissolve proteins to be converted into sols, and such sols are heated to be intertwined in networks to produce elastic ground products. 2 to 3 weight % of salt is usually added to a ground meat to produce a fish meat-origin ground product.

With a considerable amount of sugars and salt added, conventional fish meat-origin ground products are rich in saltiness and sweetness. Thus, less production of such products and improvement in such production methods are required.

In recent reports, sodium gluconate is developed as an inhibitor against heat denaturation of fish meat-origin muscular proteins in place of sorbitol (Non-Patent Document 1). While sorbitol is a sugar alcohol obtained by reducing glucose to convert aldehyde group into hydroxy group, gluconic acid is a carboxylic acid produced by oxidizing 1-carbon atom of glucose. In chemical structure, sorbitol and gluconic acid are significantly similar and both are in equilibrium with gluconolactone as a sugar in an aqueous solution. Therefore, use of sodium gluconate is examined as one type of sugars (Non-Patent Document 1).

In addition, as a salt-soluble salt, sodium gluconate is currently developed as an additive instead of salt to produce fish meat or squid meat-origin ground products (Non-Patent Document 1 or 2).

Additionally, it is reported that sodium glutamate can inhibit freeze denaturation of fish meat-origin muscular proteins (Non-Patent Document 3).

### Prior Art Documents

Non-Patent Document
Non-Patent Document 1 Nippon Suisan Gakkaishi, 65 (5), 886-891 (1999)
Non-Patent Document 2 Nippon Suisan Gakkaishi, 69 (4), 637-642 (2003)
Non-Patent Document 3 J. Biochem (Tokyo). 1975 April; 77 (4): 853-62
Non-Patent Document 4 Nippon Suisan Gakkaishi, 70 (6), 922-927 (2004)
Non-Patent Document 5 2. Shinsozai-ohyoseihin-kaihatsujigyo (New Material Application Product Development Project) (2004 Project Report by Nagasaki Prefectural Institute of Fisheries, Aug. 1, 2005)
Non-Patent Document 6 J. Food Science, vol 71, Nr. 6, 2006
Non-Patent Document 7 2006 Proceedings of the Japanese Society of Fisheries Science, Mar. 30, 2006

### SUMMARY OF THE INVENTION

### Problem to be Solved by the Invention

However, the above Non-Patent Document 1 describes a comparison between sodium gluconate and sorbitol (both as sugars) in the effect of inhibiting heat denaturation of fish meat-origin muscular proteins, showing a negligible difference therebetween, and makes no comparison therebetween in the effect of inhibiting freeze denaturation.

The above Non-Patent Document 2 provides a description of dissolution of mantle muscle proteins in Todarodes pacificus by sodium gluconate, improvement in physical properties of a ground product as a heat-induced gel and inhibition of functions of metal-dependent protease which is not inherent in a fish meat but in a Todarodes pacificus meat. However, the Non-Patent Document 2 provides no description of inhibition of heat denaturation and freeze denaturation of fish meat-origin muscular proteins by sodium gluconate.

Despite a description of an excellent freeze denaturation effect of sodium glutamate on fish meat-origin muscular proteins in the above Non-Patent Document 3, use of sodium glutamate as a denaturation inhibitor is difficult and unfavorable in the production of ground products, because it gives a particularly strong flavor as shown in conventionally-used flavor seasoning.

Meanwhile, the inventors released a report of successful production of ground products from a squid meat which is conventionally difficult to be introduced by adding organic salt such as sodium citrate (Non-Patent Document 4 to 7).

Non-Patent Documents 4 to 7 provide a description of dissolution of mantle muscle proteins in a Todarodes pacificus by organic salt such as sodium citrate, improvement in physical properties of a ground product as a heat-induced gel, and inhibition of autolysis by reducing functions of metal-dependent protease which is inherent in a Todarodes pacificus meat. Nevertheless, Non-Patent Documents 4 to 7 provide no description of inhibition of heat denaturation and freeze denaturation of muscular proteins by organic salt such as sodium citrate (Non-Patent Documents 4 to 7).

It is, therefore, one object of the present invention to solve the problems with ingredients of conventionally known inhibitors against denaturation of fish meat-origin muscular proteins such as sugars, sodium gluconate and sodium glutamate, and therefore to provide a muscular protein denaturation inhibitor which is effective only in a small addition amount because of exerting an excellent effect of heat denaturation and freeze denaturation of fish meat-origin muscular proteins, shows almost no taste as a solution, has been approved as food additives, also has been employed as a component in supplements due to its health-promoting effects, and is safe to the human body, and an additive for a fish meat ground meat capable of dissolving fish meat-origin muscular proteins and concurrently improving physical properties of a ground product, a fish meat ground meat containing the same and a method of producing the same, and a method of producing the ground product.

### Means for Solving the Problem

A muscular protein denaturation inhibitor according to the present invention comprises a citric acid salt as an active ingredient to inhibit heat denaturation and/or freeze denaturation of fish meat-origin muscular proteins. The muscular protein denaturation inhibitor in this invention is preferably used for a fish meat-origin ground meat.

An additive for a fish meat-origin ground meat according to the present invention comprises a citric acid salt as an active ingredient to inhibit heat denaturation and/or freeze denaturation of fish meat-origin muscular proteins and dissolve said fish meat-origin muscular proteins.

Formation of a heat-induced gel in a fish meat-origin ground meat requires the following conditions: no denaturation of fish meat-origin muscular proteins contained in the fish meat-origin ground meat when dissolved and no denaturation of meat-origin muscular proteins already dissolved therein. The muscular protein denaturation inhibitor in this invention comprises a citric acid salt as an active ingredient, preferably sodium citrate having a significantly strong effect of inhibiting heat denaturation and/or freeze denaturation as a citric acid salt of said active ingredient. Sodium citrate has a strong effect of dissolving fish meat-origin muscular proteins, and due to neutrality or slight alkalinity, proteins can be most stably maintained in physical properties. Also, with almost no taste in its solution, sodium citrate can provide only a distinctive flavor in the ground product without unnecessary taste added.

To prevent denaturation of muscular proteins and achieve a stable structure thereof, myofibrillar proteins contained in muscular proteins are required to keep a specific structure, which is affected by the properties of myosin. Since myosin is believed to generate a heat-induced gel, muscular proteins in this invention are preferably myofibrillar proteins, and more preferably myosin.

Next, a fish meat-origin ground meat according to the present invention is provided with said muscular protein denaturation inhibitor, but no sugars, or provided with said additive for a fish meat-origin ground meat, but neither sugars nor salt. Using the fish meat-origin ground meat as a raw material, a ground product can be produced so that an original flavor inherent in a fish meat is provided.

A method for producing a fish meat-origin ground meat according to the present invention comprises a mincing step for mincing a fish meat collected from fish as a raw material and a citric acid salt adding step for adding a citric acid salt to said fish meat, and a sugar adding step is not provided. A method for producing a fish meat-origin frozen ground meat according to the present invention comprises a mincing step for mincing a fish meat collected from fish as a raw material, a citric acid salt adding step for adding a citric acid salt to said fish meat, and a freezing step for freezing a fish meat after said mincing step and said citric acid salt adding step, and a sugar adding step is not provided. A method for producing a fish meat-origin ground meat according to the present invention comprises a mincing step for mincing a fish meat collected from fish as a raw material, a citric acid salt adding step for adding a citric acid salt to said fish meat, a freezing step for freezing a fish meat after said mincing step and said citric acid salt adding step, an unfreezing step for unfreezing a fish meat after said freezing step, and a heating step for heating a fish meat after said unfreezing step, and neither sugar adding step nor salt adding step are provided. A citric acid salt in this invention is preferably sodium citrate.

### Advantageous Effect of the Invention

A muscular protein denaturation inhibitor according to the present invention can be used as an additive which is effective only in a small addition amount because of exerting an excellent effect of inhibiting heat denaturation and freeze denaturation of fish meat-origin muscular proteins, shows almost no taste as a solution, and is safe to the human body. An additive for a fish meat ground meat according to the present invention can dissolve fish meat-origin muscular proteins in addition to said advantage, and concurrently improve the physical properties of a ground product. A fish meat-origin ground meat according to the present invention can provide a raw material of a ground product without heat denaturation or freeze denaturation of fish meat-origin muscular proteins, and a raw material of a ground product in which fish meat-origin muscular proteins are completely dissolved. A method for producing a fish meat-origin ground meat according to the present invention, a method for producing a fish meat-origin frozen ground meat, or a method for producing a fish meat-origin ground product can produce an elastic ground product in which a distinctive flavor inherent in fish meat is provided.

### BRIEF DESCRIPTION OF THE DRAWINGS

The above and other objects in this invention will be seen by reference to the description taken in connection with the drawings, in which:

FIG. 1 is a graph indicative of comparison between sodium citrate, sorbitol, sodium acetate and sodium glutamate of this embodiment in the effect of inhibiting heat denaturation of myosin. (◯ denotes data on sodium citrate in Example 1, on sodium acetate in Comparative Example 1, □ on sodium glutamate in Comparative Example 1, ▪ on sorbitol in Comparative Example 1, and vertical axis denotes logarithmic values of denaturation rate constant (K_{D}) and horizontal axis denotes the volume totaled by these compounds added (M));

FIG. 2 is a graph indicative of comparison between sodium citrate, sorbitol, sodium acetate and sodium glutamate of this embodiment in the effect of inhibiting heat denaturation of myofibrillar proteins. (◯ denotes data on sodium citrate in Example 2, on sodium acetate in Comparative Example 2, □ on sodium glutamate in Comparative Example 2, ▪ on sorbitol in Comparative Example 2, and vertical axis denotes logarithmic values of denaturation rate constant (K_{D}) and horizontal axis denotes the volume totaled by these compounds added (M));

FIG. 3 is a graph indicative of comparison between sodium citrate, sorbitol, sodium acetate and sodium glutamate of this embodiment in the effect of inhibiting freeze denaturation of myofibrillar proteins. (◯ denotes data on sodium citrate in Example 3, on sodium acetate in Comparative Example 3, □ on sodium glutamate in Comparative Example 3, ▪ on sorbitol in Comparative Example 3, and vertical axis denotes value of Ca-ATPase activity and horizontal axis denotes the volume totaled by these compounds added (mM));

FIG. 4 is a graph indicative of comparison between sodium citrate, salt, sorbitol, sodium acetate and sodium glutamate of this embodiment in solubility of myofibrillar proteins. (◯ denotes data on sodium citrate in Example 4, on sodium acetate in Comparative Example 4, ▴ on salt in Comparative Example 4, □ on sodium glutamate in Comparative Example 4, ▪ on sorbitol in Comparative Example 4, and vertical axis denotes mass of soluble proteins (relative value) of myosin as proteins collected and horizontal axis denotes the volume totaled by these compounds added (M));

FIG. 5 is a block diagram indicative of each step comprising a method for producing a fish meat-origin ground meat of this embodiment;

FIG. 6 is a block diagram indicative of each step comprising a method for producing a fish meat-origin frozen ground meat of this embodiment; and

FIG. 7 is a block diagram indicative of each step comprising a method for producing a fish meat-origin ground product of this embodiment.

### BEST MODE FOR CARRYING OUT THE INVENTION

A muscular protein denaturation inhibitor in this invention comprises a citric acid salt as an active ingredient to inhibit heat denaturation and/or freeze denaturation of fish meat-origin muscular proteins. In this invention, in a case where a citric acid salt proactively inhibits heat denaturation or freeze denaturation of fish meat-origin muscular proteins, other components comprising a muscular protein denaturation inhibitor are not particularly limited. For example, an active ingredient may be a citric acid salt it self or a solution produced by dissolving a citric acid salt in a solvent such as water.

In this embodiment, sodium citrate is used as a favorable citric acid salt, but it is not limited thereto as long as it is a citric acid salt. For example, a citric acid salt used in this invention includes potassium citrate as the same alkali metal salt, calcium citrate as an alkaline earth metal salt and copper citrate (II) as a heavy metal salt.

Hereinbelow, denaturation of proteins generally means that proteins as a biopolymer lose a higher-order structure or a lower-order structure under physiological conditions, and heat denaturation means high-temperature denaturation and freeze denaturation means low-temperature denaturation. In a case where a protein causes heat denaturation and freeze denaturation, not only higher-order structure but also primary structure can be destructed. Thermodynamic stability in three-dimensional protein structure is determined by denaturation free energy (ΔGd), a difference between free energy at natural state and denaturation state. In fact, the temperature is not a factor in denaturation. However, it is believed that freeze denaturation is normally caused at 0° C. or less. In general use, “inhibit(ion)” means a state at which a rapidly-moving object is held with external force, but in this invention, it also means that a rate of denaturation of fish meat-origin muscular proteins is reduced.

In this embodiment, determination of the effect of inhibiting denaturation of fish meat-origin muscular proteins employs a method for calculating a rate of denaturation of myosin based on disappearance of Ca-ATPase activity as bioactivity of myosin {Tohru Oizumi et al.: Gyoruikingenseni-no-kanetsuhensei-ni-taisuru-to-oyobi-toarukohru-no-hogokohka-no-teiryokosatsu (Quantitative Examination of Effect of Protecting Sugars and Sugar Alcohols from Heat Denaturation of Fish Myofibril. Journal of the Japanese Society of Fisheries Science, 47, 901-908 (1981)}. Said method in this embodiment will be described as follows by dividing into a method for determining the effect of inhibiting heat denaturation and a method for determining effect of inhibiting freeze denaturation.

Firstly, a method for determining the effect of inhibiting heat denaturation is described. Fish meat-origin myofibrillar proteins containing additives such as sodium citrate or myosin are subjected to ice cold after being heated to stop denaturation reaction. Then, changes in Ca-ATPase activity of the fish meat-origin muscular proteins heated are analyzed according to a first-order reaction equation to calculate a denaturation rate constant (K_{D}). Ca-ATPase activity is measured by preparing a reaction composition liquid composed of 0.5M KCl, 25 mM Tris-maleate (pH 7.0), 5 mM CaCl₂, 1 mM ATP and 0.2 to 0.3 mg/ml of fish meat-origin muscular proteins subjected to heating and ice cold and determining free inorganic phosphate quantitatively. Then, denaturation rate constant is calculated from Ca-ATPase activity obtained and heating time. Consequently, if the denaturation rate constant is smaller, the effect of inhibiting denaturation of fish meat-origin muscular proteins is significant, and if the denaturation rate constant is larger, the effect of inhibiting denaturation of fish meat-origin muscular proteins is small.

Next, a method for determining the effect of inhibiting freeze denaturation will be described. While the method for determining the effect of inhibiting heat denaturation is associated with time elapsed, this method determines the remaining Ca-ATPase activity under a condition of constant freezing time by changing a concentration of additives such as sodium citrate. In this case, a comparison is made not by an absolute value of denaturation rate constant but a relative value thereof. The measurement of Ca-ATPase activity is the same as above.

Also, myosin used in this embodiment is prepared by ammonium sulfate fractionation, but it is not particularly limited thereto. For example, the myosin can be prepared using known methods such as gel filtration chromatography and ion-exchange chromatography. A method for determining the effect of inhibiting denaturation of proteins is not particularly limited to said method. For example, using structural analysis by X-ray diffraction method and nuclear magnetic resonance (NMR) spectroscopy, along with other effective methods, the effect of inhibiting denaturation of muscular proteins can be examined.

Fish species in this invention are not particularly limited to carp meat used in this embodiment, but include any fish species such as freshwater fish, saltwater fish, brackish water fish, and fish which can inhabit on land like tideland and marsh.

In this embodiment, the effect of inhibiting denaturation is evaluated by making a comparison using myosin and then another comparison using myofibrils. This evaluation is based on confirmation of an effect on stability on myosin itself to evaluate an effect on stability of myofibrils as muscle model. Specifically in this invention, muscular proteins are not particularly limited to myofibrillar proteins or myosin, but may be, for example, actin and actomyosin.

Next, an additive for a fish meat-origin ground meat in this invention has an effect of dissolving fish meat-origin muscular proteins. In general concept, dissolution of proteins corresponds to monomolecular dispersion of proteins as opposed to denaturation of proteins. Specifically in this embodiment, proteins are dispersed monomolecularly by sodium citrate to which fibrous myofibrillar proteins or myosin is added to form sols. In this invention, when a citric acid salt is added to fish meat-origin muscular proteins, the citric acid salt dissolves fish meat-origin muscular proteins while inhibiting heat denaturation and freeze denaturation thereof. It is suggested that the citric acid salt in this invention has effects of inhibiting denaturation of muscular proteins and dissolving muscular proteins.

Next, in this invention, a method for producing a fish meat-origin ground meat, a method for producing a fish meat-origin frozen ground meat and a method for producing a fish meat-origin ground product each comprise a mincing step for mincing a fish meat collected from fish as a raw material and a citric acid salt adding step for adding a citric acid salt to said fish meat. Since heat denaturation of muscular proteins is mainly caused by frictional heat from the mincing treatment, said citric acid salt adding step is not particularly limited to a step after said mincing step. For example, said citric acid salt adding step may be performed prior to said mincing step, as the mincing treatment comes after adding a citric acid salt to a fish meat collected from fish as a raw material. Said mincing step may be provided with said citric acid salt adding step, as a citric acid salt is added in the mincing treatment.

In this invention, a method for producing a fish meat-origin ground meat and a method for producing a fish meat-origin frozen ground meat comprise no sugar adding step, and a method for producing a fish meat-origin ground product comprises neither sugar adding step nor salt adding step. No preparation of said sugar adding step or said salt adding step means no addition of sugars or salt ranging from a step for producing a fish meat-origin ground meat, a step for producing a fish meat-origin frozen ground meat to a step for producing a fish meat-origin ground product in this invention.

A step for producing a fish meat-origin ground meat, a step for producing a fish meat-origin frozen ground meat and a step for producing a fish meat-origin ground product each include a step for washing fish, a step for descaling, a step for treating a fish body such as fillet treatment, a water-exposing step, a step for removing contaminants, a step for dehydrating and a step for grinding, but a method for producing a fish meat-origin ground meat, a method for producing a fish meat-origin frozen ground meat and a method for producing a fish meat-origin ground product in this invention each may comprise all, any one or none of these steps.

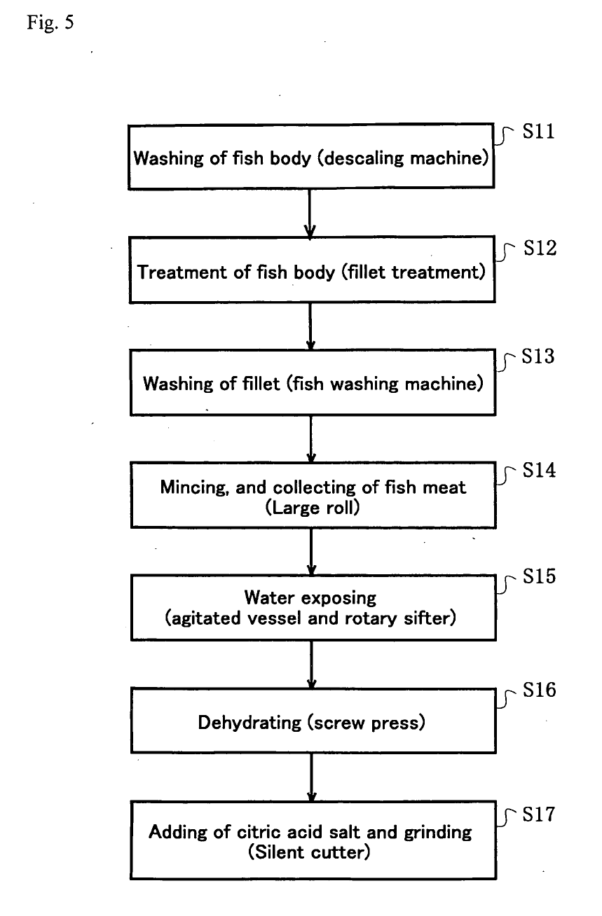

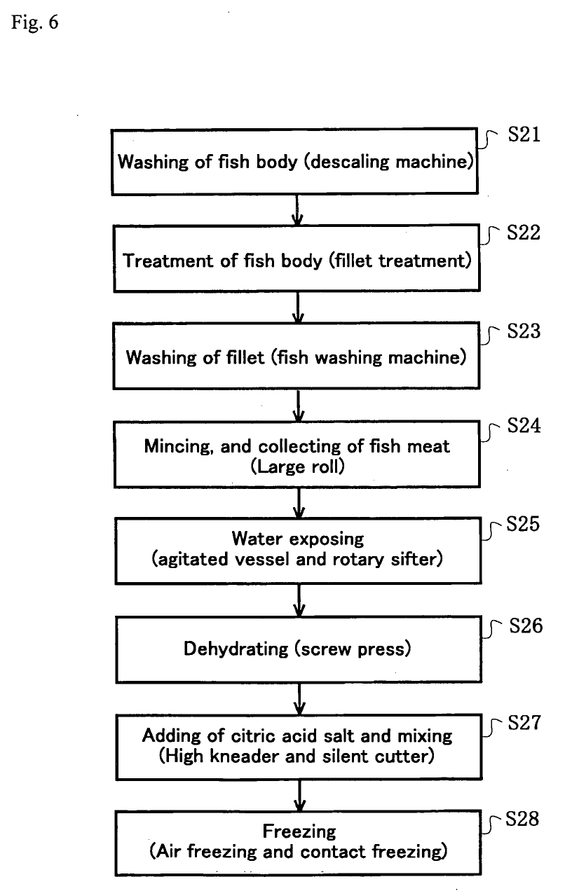

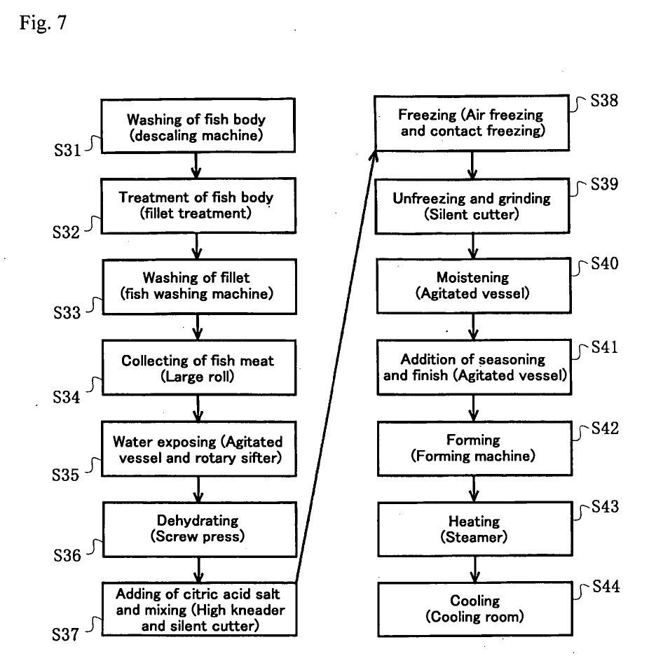

A freezing step for freezing said fish meat in a method for producing a fish meat-origin frozen ground meat and a method for producing a fish meat-origin ground product in this invention is provided after said mincing step and said citric acid salt adding step, and may be through any step provided prior to said freezing step and after said mincing step and said citric acid salt adding step. An unfreezing step for unfreezing a fish meat after said freezing step in a method for producing a fish meat-origin ground product in this invention and a heating step for heating a fish meat after said unfreezing step may be through any step provided prior to said unfreezing step and after said freezing step, or may be through any step provided prior to said heating step and after said unfreezing step. Each step in a method for producing a fish meat-origin ground meat, a method for producing a fish meat-origin frozen ground meat and a method for producing a fish meat-origin ground product in this embodiment is shown in FIGS. 5 to 7.

Examples of a muscular protein heat denaturation inhibitor and a muscular protein freeze denaturation inhibitor according to the present invention will be described. The scope of the present invention is not particularly limited to these examples.

### EXAMPLE

### Example 1

### Effect of Inhibiting Heat Denaturation of Sodium Citrate on Muscular Protein Myosin

After myofibrils were prepared from dorsal meat of carp (Cyprinus carpio) according to a method by Noboru Katoh et al. {Noboru Katoh et al.: Gyruikingenseni-ATPase-no-seikagakuteki-kenkyu (Biochemical Research of Fish Myofibrillary ATPase). Journal of the Japanese Society of Fisheries Science, 43, 857-867 (1977)}, the myofibrils were dissolved in 0.5M KCl in the presence of ATP-Mg and 40 to 55% ammonium sulfate fraction was used as myosin by ammonium sulfate fractionation. After the myosin obtained was heated at 35° C. in a constant-temperature water bath in the presence of sodium citrate (Wako Pure Chemical Industries, Ltd) and part thereof was subjected to ice cold as time was elapsed, the product was added to 0.5M KCl, 25 mM Tris-maleate (pH 7.0), 5 mM CaCl₂ and 1 mM ATP to prepare a reaction composition liquid to be reacted at 25° C. After 5% perchloric acid was added thereto to stop the reaction and filter the product, free inorganic phosphate was subjected to colorimetric determination by phosphomolybdic acid method to calculate a logarithmic value of denaturation rate constant.

### Comparative Example 1

### Effect of Inhibiting Heat Denaturation of Sorbitol, Sodium Acetate and Sodium Glutamate on Muscular Protein Myosin

Using the same method as Example 1, a logarithmic value of denaturation rate constant was calculated when sorbitol, sodium acetate or sodium glutamate (all produced by Wako Pure Chemical Industries) was added.

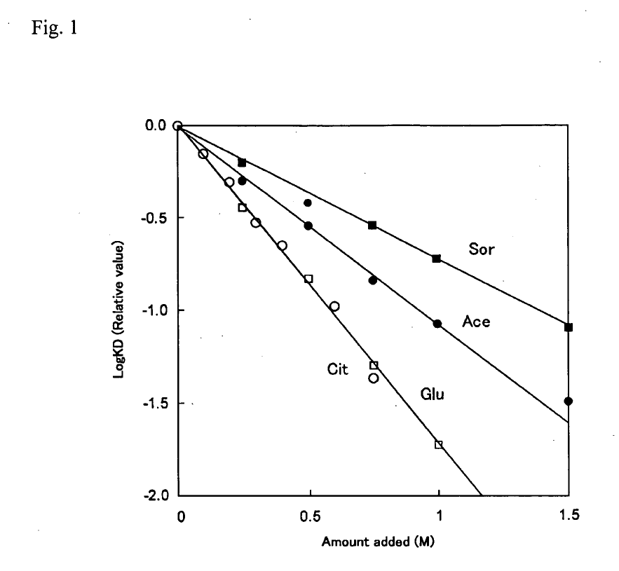

FIG. 1 shows the results of Example 1 and Comparative Example 1. In each case where a compound was added, rate of denaturation of myosin declined, showing each compound has an effect of stabilizing the myosin. As shown in FIG. 1, when sodium citrate, sorbitol, sodium acetate and sodium glutamate were each added by 1M, the rate of denaturation declined by 1.7, 0.7, 1.1, and 1.7, respectively. This indicates that sodium citrate has an effect of stabilizing myosin, the extent of which is equivalent to sodium glutamate having the most significant effect of stabilizing proteins.

### Example 2

### Effect of Inhibiting Heat Denaturation of Sodium Citrate on Myofibrillar Proteins

After myofibrils obtained by the same method as Example 1, which are stabilized by actomyosin, were heated in a constant-temperature water bath at 42° C., and part thereof was taken as time was elapsed and subjected to ice cold in the presence of sodium citrate (same as above), the product was added to 0.5M KCl, 25 mM Tris-maleate (pH 7.0), 5 mM CaCl₂ and 1 mM ATP to prepare a reaction composition liquid to be reacted at 25° C. After 5% perchloric acid was added thereto to stop the reaction and filter the product, free inorganic phosphate was subjected to colorimetric determination by phosphomolybdic acid method to calculate a logarithmic value of denaturation rate constant.

### Comparative Example 2

### Effect of Inhibiting Heat Denaturation of Sorbitol, Sodium Acetate and Sodium Glutamate on Myofibrillar Proteins

Using the same method as Example 2, logarithmic values of denaturation rate constant were calculated when sorbitol, sodium acetate or sodium glutamate (same as above) was added.

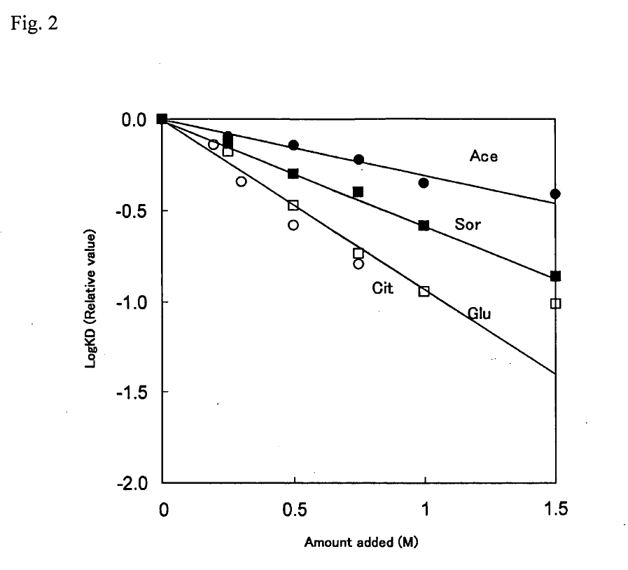

FIG. 2 shows the results of Example 2 and Comparative Example 2. In each case where a compound was added, rate of denaturation of myofibrillar proteins declined, showing each compound has an effect of stabilizing myofibrillar proteins. As shown in FIG. 2, when sodium citrate, sorbitol, sodium acetate and sodium glutamate were each added by 1M, the rate of denaturation declined by 0.9, 0.6, 0.3 and 0.9, respectively. With an overall small effect compared to a case where myosin was used, it is suggested that sodium citrate has an effect of stabilizing myofibrillar proteins, the extent of which is equivalent to sodium glutamate and about twice that of sorbitol.

According to the above Non-Patent Document 1, since sodium gluconate can inhibit heat denaturation of myofibrillar proteins about 1.5 times sorbitol, it is indicated that sodium citrate has an effect of stabilizing myofibrillar proteins at least 1.3 times sodium gluconate.

### Example 3

### Effect of Inhibiting Freeze Denaturation of Sodium Citrate on Myofibrillar Proteins

Myofibrils obtained by the same method as Example 1 were subjected to cryopreservation at −20° C. in the presence of sodium citrate (same as above). The myofibrils were taken approximately 1 month later and added to 0.5M KCl, 25 mM Tris-maleate (pH 7.0), 5 mM CaCl₂ and 1 mM ATP to prepare a reaction composition liquid to be reacted at 25° C. After 5% perchloric acid was added thereto to stop the reaction and filter the product, free inorganic phosphate was subjected to colorimetric determination by phosphomolybdic acid method to calculate Ca-ATPase activity and analyze the extent of denaturation observed from the decline of Ca-ATPase activity.

### Comparative Example 3

### Effect of Inhibiting Freeze Denaturation of Sorbitol, Sodium Acetate and Sodium Glutamate on Myofibrillar Proteins

Using the same method as Example 3, Ca-ATPase activity was calculated when sorbitol, sodium acetate or sodium glutamate (same as above) was added thereto.

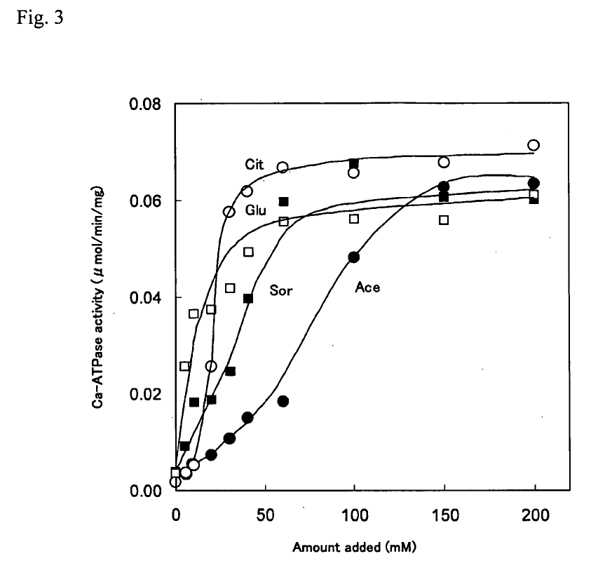

FIG. 3 shows the results of Example 3 and Comparative Example 3. It was found that when none of these compounds is not added to myofibrillar proteins, almost no Ca-ATPase activity was observed, resulting in significant denaturation observed. Meanwhile, it was found that when each compound was added to myofibrillar proteins, the activity becomes smaller as the volume of each compound is increased. As shown in FIG. 3, sodium acetate provides the smallest effect of inhibiting freeze denaturation, followed by sorbitol. On the other hand, both sodium citrate and sodium glutamate demonstrated the strongest effect of inhibiting freeze denaturation.

### Example 4

### Solubility of Sodium Citrate on Myofibrillar Proteins

After myofibrils obtained by the same method as Example 1 were suspended in a buffer solution (0.1 M salt and 20 mM Tris-HCl (pH 7.5)) to prepare a myofibrillary suspension to be stored at 0° C. for 6 to 24 hours, an absorbance of said suspension at 350 nm was measured and defined as turbidity. Then, sodium citrate was added to said suspension, the product was swiftly subjected to centrifugal separation (KOKUSAN, 20,000×g, 25 minutes) to measure the volume of proteins collected in supernatants by colorimetric method and define a relative value to protein concentration of said suspension prior to centrifugal separation as a volume of soluble proteins (relative value).

### Comparative Example 4

### Solubility of Sorbitol, Salt, Sodium Acetate and Sodium Glutamate on Myofibrillar Proteins

Using the same method as Example 4, the volume of soluble proteins was determined when sorbitol, salt, sodium acetate or sodium glutamate (Wako Pure Chemical Industries) was added.

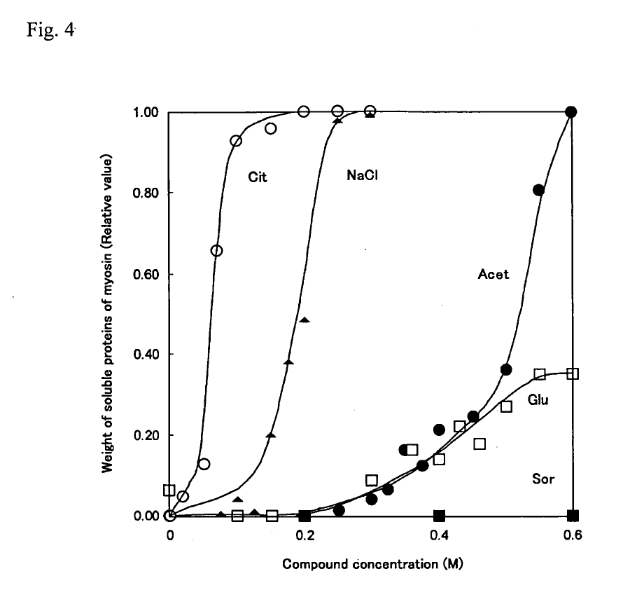

FIG. 4 shows the results of Example 4 and Comparative Example 4. Each compound which was not added to myofibrillar proteins contained insoluble proteins. As shown in FIG. 4, despite the addition of sorbitol with a significantly high concentration of 0.6M, the proteins were not dissolved at all. It was found that the proteins were completely dissolved in cases of addition of up to 0.1M sodium glutamate, approximately 0.2M salt and 0.6M sodium acetate, while addition of 0.6M sodium glutamate caused incomplete dissolution of under 40% proteins. It was found that sodium citrate has a significantly high effect of dissolving proteins, compared to salt.

In order to evaluate whether myosin and myofibrillar proteins can be used as a raw material of a fish meat-origin ground product in the form of a fish meat-origin ground meat or a fish meat-origin frozen ground meat, based on Ca-ATPase activity thereof, known evaluation methods {Takayoshi Kawashima, Kenich Arai et al.: suketohdara reitohsurimichuh-no actomyosin-ganryo-to-kamaboko-no-dansei-tono-kankei-nitsuite (Relationship between Actomyosin Content in Theragra Chalcogramma Frozen Ground Meat and Elasticity of Kamaboko Fish Paste). Journal of the Japanese Society of Fisheries Science, 39, 1201-1209 (1973), S. Koseki, K. Konno, et al.: Quality evaluation of frozen ground meat by using pH stat for ATPase assay. Fish. Sci. 71, 388-396 (2005)} can be used. Using sodium citrate, h eat denaturation and freeze denaturation are inhibited, or myosin and myofibrillar proteins dissolved in this embodiment can be employed as a raw material of a fish meat-origin ground product in the form of a fish meat-origin ground meat and a fish meat-origin frozen ground meat.

From the above considerations in this embodiment, the present invention can provide the following advantages:

1. A muscular protein denaturation inhibitor and an additive for a fish meat-origin ground meat which include health-promoting effects and are safe to the human body;
2. A fish meat-origin ground product which is less sweet, less salty and can provide a distinctive flavor inherent in fish meat;
3. A relatively inexpensive elastic ground product which can inhibit heat denaturation and freeze denaturation of muscular proteins of a fish meat-origin ground meat only in a small addition amount and dissolve muscular proteins.

1. A muscular protein denaturation inhibitor to inhibit heat denaturation and/or freeze denaturation of fish meat-origin muscular proteins, comprising a citric acid salt as an active ingredient.

2. The muscular protein denaturation inhibitor as set forth in claim 1, wherein the muscular protein denaturation inhibitor is used for a fish meat-origin ground meat.

3. The muscular protein denaturation inhibitor as set forth in claim 1, wherein a citric acid salt is sodium citrate.

4. The muscular protein denaturation inhibitor as set forth in claim 1, wherein a muscular protein is a myofibrillar protein.

5. The muscular protein denaturation inhibitor as set forth in claim 4, wherein a myofibrillar protein is myosin.

6. An additive for a fish meat-origin ground meat to inhibit heat denaturation and/or freeze denaturation of fish meat-origin muscular proteins and dissolve said fish meat-origin muscular proteins, comprising a citric acid salt as an active ingredient.

7. The additive for a fish meat-origin ground meat as set forth in claim 6, wherein a citric acid salt is sodium citrate.

8. The additive for a fish meat-origin ground meat as set forth in claim 6, wherein a muscular protein is a myofibrillar protein.

9. The additive for a fish meat-origin ground meat as set forth in claim 8, wherein a myofibrillar protein is myosin.

10. A fish meat-origin ground meat, comprising the muscular protein denaturation inhibitor as set forth in any one of claims 1 to 5 and no sugars.

11. A fish meat-origin ground meat, comprising the additive for a fish meat-origin ground meat as set forth in any one of claims 6 to 9 and neither sugars nor salt.

12. The fish meat-origin ground meat as set forth in claim 10, wherein a ground meat is a frozen ground meat.

13. A method of producing a fish meat-origin ground meat, comprising the steps of:
a mincing step for mincing a fish meat collected from fish as a raw material; and
a citric acid salt adding step for adding a citric acid salt to said fish meat,
wherein a step for adding sugars is not provided.

14. The method of producing a fish meat-origin ground meat as set forth in claim 13, wherein a citric acid salt is sodium citrate.

15. A method of producing a fish meat-origin frozen ground meat, comprising the steps of:
a mincing step for mincing a fish meat collected from fish as a raw material;
a citric acid salt adding step for adding a citric acid salt to said fish meat; and
a freezing step for freezing a fish meat after said mincing step and said citric acid salt adding step,
wherein a step for adding sugars is not provided.

16. The method of producing a fish meat-origin frozen ground meat as set forth in claim 15, wherein a citric acid salt is sodium citrate.

17. A method of producing a fish meat-origin ground product, comprising the steps of:
a mincing step for mincing a fish meat collected from fish as a raw material;
a citric acid salt adding step for adding a citric acid salt to said fish meat;
a freezing step for freezing a fish meat after said mincing step and said citric acid salt adding step;
an unfreezing step for unfreezing a fish meat after said freezing step; and
a heating step for heating a fish meat after said unfreezing step,
wherein a step for adding sugars and a step for adding salt are not provided.

18. The method of producing a fish meat-origin ground product as set forth in claim 17, wherein a citric acid salt is sodium citrate.

19. A method for inhibiting heat denaturation and/or freeze denaturation of fish meat-origin muscular proteins, adding a citric acid salt as an active ingredient to fish meat-origin muscular proteins.

20. The method as set forth in claim 19, wherein the muscular protein denaturation inhibitor is used for a fish meat-origin ground meat.

21. The method as set forth in claim 19, wherein a citric acid salt is sodium citrate.

22. The method as set forth in claim 19, wherein a muscular protein is a myofibrillar protein.

23. The method as set forth in claim 22, wherein a myofibrillar protein is myosin.
